SEQUENCE LISTING (6 sequences)

SEQ ID NO: 1 - protein, 247 aa, Homo sapiens
  NC1
MQVVKEQVMRALTTKPSSLDQFKSKLQNLSYTEILKIRQSERMNQEDFQSRPILELKEKIQPEILELIKEERLNRLVEGTCFRKLNARRRQDKFWYCRKSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRDDLDTLLSMEIKLRLLDLENIQIPDAPPPIPKEPSNYDFVYDCN

SEQ ID NO: 2 - protein, 679 aa, Homo sapiens
  NC2
MDRVTRYPILGIPQAHRGTGLVLDGDTSYTYHLVCMGPEASGWGQDEPQTWPTDHRAQQGVQRQGVSYSVHAYTGQPSPRGLHSENREDEGWQVYRLGARDAHQGRPTWALRPEDGEDKEMKTYRLDAGDADPRRLCDLERERWAVIQGQAVRKSSTVATLQGTPDHGDPRTPGPPRSTPLDDNVVDREQIDFLAARQQFLSLEQANKGAPHSSPARGTPAGTTPGASQAPKAFNKPHLANGHVVPIKPQVKGVVREENKVRAVPTWASVQVVDDPGSLASVESPGTPKETPIEREIRLAQEREADLRDQRGLRQATDHQELVEIPTRPLLTKLSLITAPRRERGRPSKYVQRDIVQETQREEDHRREGKHVGRASTPDWVSEGPQPGLRRALSSDSILSPAPDARAADPAPEVRKVNRIPPDAYQPYLSPGTPQLEFSAFGAFGKPSSLSTAEAKAATSPKATMSPRHLSESSGKPLSTKQEASKPPRGCPQANRGVVRWEYFRLRPLRFRAPDEPQQAQVPHVWGWEVAGAPALRLQKSQSSDLLERERESVLRREQEVAEERRNALFPEVFSPTPDENSDQNSRSSSQASGITGSYSVSESPFFSPIHLHSNVAWTVEDPVDSAPPGQRKKDQWYAGINPSDGINSEVLEAIRVTRHKNAMAERWESRIYASEEDD

SEQ ID NO: 3 - protein, 119 aa, Homo sapiens
  NC3
MADGLFRRRPWGLEQIRPDPESEGLFDKPPPEDPPAARGPRSASAAGKKAGRRAGGRAQGGRAGQPPKAASRPPPKKEAPPLDEGCYLDHFPHLSIFIYAAIAFSITSCIFTYIHLQLA

SEQ ID NO: 4 - DNA, 2109 bp, Homo sapiens
  CDS (174)...(904): NC1
atgttcacatggctcaactggaaacctgtttcatgaacaagcttactcaggaaccatctggtggtattccagcacattgttcttcagggggacgactctaagtcgctttgtggtggcagcagcttagaatcagtatttgtggttgggaaagatggacttacgggagcttggtaatgcaggtggtgaaggagcaggttatgagagcacttacaaccaagcctagctccctggaccagttcaagagcaaactgcagaacctgagctacactgagatcctgaaaatccgccagtccgagaggatgaaccaggaagatttccagtcccgcccgattttggaactaaaggagaagattcagccagaaatcttagagctgatcaaacagcaacgcctgaaccgccttgtggaagggacctgctttaggaaactcaatgcccggcggaggcaagacaagttttggtattgtcggctttcgccaaatcacaaagtcctgcattacggagacttagaagagagtcctcagggagaagtgccccacgattccttgcaggacaaactgccggtggcagatatcaaagccgtggtgacgggaaaggactgccctcatatgaaagagaaaggtgcccttaaacaaaacaaggaggtgcttgaactcgctttctccatcttgtatgactcaaactgccaactgaacttcatcgctcctgacaagcatgagtactgtatctggacagatggactgaatgcgctactcgggaaggacatgatgagcgacctgacgcggaatgacctggacaccctgctcagcatggaaatcaagctccgcctcctggacctggaaaacatccagatccctgacgcacctccgccgattcccaaggagcccagcaactatgacttcgtctatgactgtaactgaagtggccgggcccagacatgccccttccaaaactggaacacctagctaacaggagagaggaatgaaaacacacccacgccttggaaccgtcctttggtaaagggaagctgtgggtccacattcccttcagcatcacctctagccctggcaactttcagcccctagctggcatcttgctcaccgccctgattctgttcctcggctccactgcttcaggtcacttcccatggctgcagtccactggtgggacaagagcaaagcccactgccagtaagaaggccaaagggcccttccatcctagccctctgcaggcatgcccttccttcccttgggcaggaaagccagcagccccagactgcccaaaaacttgcccaccagaccaagggcagtgccccaaggcccctgtctggaggaaatggcctagctatttgatgagaagaccaaaccccacatcctcctttcccctctctctagaatcatctcgcaccaccagttacacttgaattaagatctgcgctcaaatctcctcccacctctctccctgcttttgccttgctctgttcctctttggtcccaagagcagcagccgcagcctcctcgtgatcctccctagcataaatttcccaaacagtccacaggtcccatgcccactttgcgtctgcactgtgatcgtgacaaatcttccctcctcaccagctagtctggggtttcctctccctgccccaggccagaactgccttcttcatttccacccacgctcccagcctcttagctgaaagcacaaatggtgaaatcagtagtctcgctccatctctaatagactaaacctaaatgcctctaggacggactgttgctatccaagcgtttggtgttaccttctcctgggaggtcctgctgcaactcaagttccacaggatggtcaagctgtcagacatccaagtttacatcattgtaattattactggtatttacaatttgcaagagttttgggttagtttttttttttttttttttgctttgtttttgtacaaaagagtctaacattttttgccaaacagatatatatttaatgaaaagaagagatacataaatgtgtgaatttccagttttttttttaattattttaatcccaaacatcttcctgaaaataacattcccttaaacatgctgtggaataaaatggattgtgatg

SEQ ID NO: 5 - DNA, 2846 bp, Homo sapiens
  CDS (79)...(2115): NC2
tttccttctcctccctcagtaagcccagaggtctccaccccacgggaggaaggctgaggccaagaccccggaagagatatggaccgcgtgaccagataccccatcctgggcatccctcaggcacaccgtggcaccggcctggtgctggatggagacaccagctacacataccatctggtgtgcatgggccccgaggccagcggctggggccaggatgagccgcagacatggcccactgaccacagggcccagcagggcgtgcagaggcagggggtgtcctacagcgtgcatgcctacactggccagccgtccccacgggggctccactcggagaacagggaggatgagggttggcaggtttaccgcctgggcgccagggatgcccaccagggacgtccaacatgggcactccgcccagaggacggggaggacaaggagatgaagacctaccgcctggatgctggggacgctgaccccaggaggctgtgtgacctggagcgggagcgctgggccgtcatccagggccaggcagtcaggaagagcagcaccgtggccacgctccagggcactcctgaccacggagaccccaggacccccggcccacctcggtccacgcccctggaggagaacgtggttgacagggagcagattgacttcctggcagcgagacagcagttcctgagtctggagcaggcgaacaagggggcccctcatagctccccggccagggggacccctgcaggcacaaccccaggggccagccaggcccccaaggccttcaacaagccccacctggccaacgggcacgtggttcccatcaagccccaggtgaagggggtggtcagggaagagaacaaggtgcgtgctgtgcccacctgggccagtgtccaagttgtggatgaccctggctccttggcctcagtggagtccccggggacccccaaggagacgcccatcgagcgggagatccgtctggctcaggagcgtgaggcagacctgcgagagcagagggggcttcggcaggcaaccgaccaccaggagctggtggaaatccccaccaggccgctgctgaccaagctgagcctgatcacagccccacggcgggagagagggcgcccgtccctctacgtgcagcgggacatagtacaggagacacagcgtgaggaagaccaccggcgggagggcctgcacgtgggccgggcgtccacacccgactgggtctcggagggtccccagcccggactccggagagccctcagctcagattccatcctcagcccggccccagatgcccgtgcggccgacccagctccagaagtgaggaaggtgaaccgcatcccacctgatgcctaccagccgtacctgagccccgggaccccccagctagaattctcagccttcggagcattcggcaagcccagcagtctctccacagcggaggccaaggctgcgacttcaccaaaggccacgatgtccccgaggcatctctcagaatcctctggaaaacccctgagcacaaagcaagaggcatcgaagccccctcggggatgcccgcaagccaacaggggtgtcgtgcggtgggagtacttccgcctgcgtcctctgcggttcagggccccagacgagccccagcaggcccaagtcccccatgtctggggctgggaggtggctggggcccctgcactgaggctgcagaagtcccagtcatctgatctgctggaaagggagagggagagtgtcctgcgccgggagcaagaggtggcagaggagcggagaaatgctctcttcccagaggtcttctccccaacgccagatgagaactctgaccagaactccaggagctcctcccaggcatccggcatcacgggcagttactcggtgtctgagtctcccttcttcagccccatccacctacactcaaacgtggcgtggacagtggaagatccagtggacagtgctcctcccgggcagagaaagaaggagcaatggtacgctggcatcaacccctcggacggtatcaactcagaggtcctggaagccatacgggtgacccgtcacaagaacgccatggcagagcgctgggaatcccgcatctacgccagtgaggaggatgactgagcctcgggatggggcgcccaccccctgccctgccctgaccctcgtgggaactgccaagaccatcgccaagcccccaccctaggaaatgggtcctaggtccaggatccaagaaccacagctcatctgccaacaatcccaccatgggcacatttgggactgttgggtttttcgtttccgtttctatcttcctttagaaatgtttctgcctttggggtctaaagcttttggggatgaaatgggacccctgctgattctttctgcttctaagactttgccaaatgccctgggtctaagaaagaaagagacccgctcctccactttcaggtgtaatttgcttccgctagtctgagggcagagggaccggtcaaagagggtggcacagatcgcagcaccttgaggggctgcgggtctgagggaggagacactcagctcctccctctgagaagtcccaagctgagaggggagacctgcccctttccaaccctgggaaaccatccagtctgagggaggaggccaaactcccagtgctgggggtccctgtgcagccctcaaacccttcaccttggtgcacccagccacacctggtggacacaaagctctcacatcgataggatcccatgaggatggtccccttcacctgggagaaaagtgacccagtttaggagctggaggggggtctttgtcccccacccccaaactgccctgaaataaacctggagtgagctgcc

SEQ ID NO: 6 - DNA, 1556 bp, Homo sapiens
  CDS (28)...(384): NC3
ctgacccacctacccgcgatcctgcccatggctgacgggctctttcggcgcagaccctggggtctccagcagattcgcccggaccccgagtccgaaggcctgtttgacaagcctcccccggaagaccctcccgctgcccgcgggcccaggtcggcgtcggccgcgggcaagaaggctggtcggcgcgcgggcgggagggcgcaggggggccgcgccgggcagcccccgaaggccgcatcgcgccccccgcccaagaaggaggcgcctccactggacgagggctgctatctcgaccattttccgcacctctccatcttcatctacgcagccatcgccttctccatcacctcctgcatctttacctatatccatttacagcttgcctgagtggccagcgcgggacggggtgggcgcaggaccgagcggggagggaaagggaaaacggggctcggcattttgtgttttagaacagcgctgcacccccttcatgtagctttcgatgcttgtttctttccgtctttgttgtcactatctttgtctatcagtacgaaagtacaaagtagctgccggcaatgaaataggggtgctgtttgcacctgcaggttaggggtggaggcgtttagaattttggggtgtgattgagccccgtttataattagaatgcccctggacccctaccactctgtgacgtgggggcacgcgcagggatcccatcattttgtgtttggggagctcagagtgcgcccaatcttggaatctttaagggatgagccagacccagacccgcggccttctagagagggtccggcagggagggtcggcgccctggcccggggtgggccggagccctgtgatgctgcatcgcccccaggaggagccagctgtgccccagagttggcgcggccgagagaggacaagagcgcgcagcaggcgaagctggagggcgggactcggtaagtggcgttcgtcggggtgtcgtgctgcgcccccaggggctccggctgaccacgactgtgtgtttttcctgccttagactttgttgtcgctgcccggaggagtcgagactggtacccggaggagctgtctcaccaggagaccacgtcctggaagtgtccgggactcgcgggcggtgtggctgcagaccccgccggcacgcaggcccagagctggcgcactcctgaggatgagactctgggggccctagccggggtccacgggagggctgtccttggggactctaggatggcttcgttctggcccggctcacttctggagctgtgagacccaagacaaaaggggctgagggatttctcattgacaagggttcgtgcgggaaaaccacatgatccctgggatttgtcatcttaagactcaaaaggcttaataccaggaaccaccttggcaagatattttacccaccggccatctctgtttactcatgaatgttaaatgttaaaacgcagcgctctaaccctgcatattatttacttgcaaatgtctgtaatctgtaattgtgatgcctctgatggaataaattatctttttcagtctcctct

(19) United States
(12) Patent Application Publication
(10) Pub. No.: US 2006/0057645 A1
(43) Pub. Date: Mar. 16, 2006

(54) Novel genes

(76) Inventors: Hideo Niwa, Akashi-shi (JP); Kenji Yamashita, Takamatsu-shi (JP)

Correspondence Address: Brinks Hofer Gilson & Lione, Po Box 10395, Chicago, IL, 60610, US

(21) Appl. No.: 10/507,876

(22) PCT Filed: Mar. 6, 2003
(86) PCT No.: PCT/JP03/02620

§ 371 Date: May 5, 2005

(30) Foreign Application Priority Data
Mar. 15, 2002 (JP) 2002-71592

(51) Int. Cl. G01N 33/567; C07H 21/04; C12N 5/08; C07K 16/26

(52) U.S. Cl. 435/7.2; 435/366; 435/320.1; 530/388.24; 536/23.5

(57) ABSTRACT

The object of the present invention is to detect and select insulin-producing cells capable of proliferating, and further to differentiate/proliferate insulin-producing cells and precursory thereof, or cells related thereto. The present invention was made by finding three species of novel genes by detecting genes specifically expressed in pancreases of PHHI patients which are regarded as a model of spontaneous proliferation of pancreatic β cells, and searching for base sequence data bases. According to the present invention, proliferating insulin-producing cells can be detected using these genes, gene products or gene sequences by, for example, Northern analysis or RT-PCR. Furthermore, these genes can be differentiated into insulin-producing cells by introducing these genes into appropriate cells in genetic engineering manner.

### TECHNICAL FIELD

The present invention relates to a technology of detecting proliferating insulin-producing cells from a tissue or cell population constituted of a plurality of cell species. The invention further relates to a technology of proliferating pancreatic β cells, which are insulin-producing cells, and cells precursory thereof or cells related to pancreatic β cells, for example nerve cells and the like.

### BACKGROUND ART

Pancreatic β cells are the only organ producing insulin, which is a peptide hormone capable of lowering the blood sugar level. When the insulin-producing ability of pancreatic β cells is impaired from some or other cause, it becomes impossible to maintain the blood sugar level within a normal range, resulting in the onset of diabetes. Transplanting cells capable of producing insulin into patients with diabetes whose insulin productivity has been impaired serves as a fundamental therapy for restoring the insulin productivity in them and maintaining their blood sugar levels within a normal range.

Conceivable as the source of supply of insulin-producing cells are dead body-derived pancreases, or cells capable of producing insulin differentiated from embryonic stem cells (ES cells) or mesenchymal stem cells, which are expected to be put into practical use in the near future. Embryonic stem cells are cells derived from the blastocyst inner cell mass and are capable of differentiating into almost all tissues or cells. Mesenchymal stem cells are pluripotent cells found in the bone marrow, blood, corium, periosteum, etc. It has been shown in recent years that these cells can be artificially caused to differentiate into such functional cells as insulin-producing cells, nerve cells and myocardial cells in vitro and in vivo. However, since the supply of such tissues or cell populations is limited, it is considered difficult to secure a number of cells sufficient to treat patients with diabetes. Therefore, proliferating insulin-producing cells or cells serving as the source of supply thereof is demanded.

A technique which possibly produce such effect comprises causing a cell differentiating/proliferating factor, such as HGF (hepatocyte growth factor), Reg protein or betacellulin, to act on such supply source cells (Otonkoski et al., Diabetes, vol. 43, pp. 947-953, 1994; Watanabe et al., Proc. Natl. Acad. Sci., vol. 91, pp. 3589-3592, 1994; Yamamoto et al., Diabetes, vol. 49, pp. 2021-2027, 2000). However, any factor capable of being applied for practical treatment purposes has not been found as yet.

Furthermore, if success is achieved in proliferating a cell population containing insulin-producing cells using such a cell differentiation/proliferation factor, it will be desired that truly effective cells alone, namely insulin-producing cells capable of proliferating alone, among the cell population be used for therapeutic purposes. Any method effective in selecting such cells has not been established as yet.

### SUMMARY OF THE INVENTION

It is an object of the present invention to provide a method of detecting the desired cells, namely insulin-producing cells capable of proliferating, from among a cell population containing a sufficient number of insulin-producing cells for therapeutic purposes using a cell differentiation/proliferation factor and, further, a method of selecting such cells and to provide a method of more efficiently differentiating/proliferating pancreatic β cells, which are insulin-producing cells, or cells related thereto, for example nerve cells and the like, for the treatment of diseases due to absolute insufficiency of a physiologically active substance to be produced in vivo, for example diabetes.

The present invention relates to a novel gene specifically expressed in the pancreas of PHHI patients, and the protein translated from that gene, and to a method of utilizing the same.

The protein of the invention is

(1) a protein

which comprises the amino acid sequence shown under SEQ ID NO:1, 2 or 3 or

(2) a protein

which has the amino acid sequence shown under SEQ ID NO:1, 2 or 3.

The novel gene of the invention is

(1) a DNA

which codes for the protein comprising the amino acid sequence shown under SEQ ID NO:1, 2 or 3,

(2) a DNA

which comprises the base sequence shown under SEQ ID NO:4, 5 or 6, or

(3) a DNA which comprises the base sequence from the 174th to 904th base in SEQ ID NO:4,

a DNA which comprises the base sequence from the 79th to 2115th base in SEQ ID NO:5, or

a DNA which comprises the base sequence from the 28th to 384th base in SEQ ID NO:6, or

(4) a DNA comprising part of the base sequence shown under SEQ ID NO:4, 5 or 6

which has at least the base sequence of DNA of the partial sequence region defined above under (3).

As the method of utilizing the novel gene and protein according to the invention, there may be mentioned

(1) a method of detecting proliferating insulin-producing cells

which comprises using the novel DNA of the invention,

(2) a transformant cell

which is obtainable by using a vector containing the novel DNA inserted therein,

(3) a method of differentiating embryonic stem cells or mesenchymal stem cells

which comprises introducing the novel DNA into a primary cell or an established cell line comprising embryonic stem cells or mesenchymal stem cells, and

(4) a method of proliferating embryonic stem cells or mesenchymal stem cells

which comprises introducing the novel DNA into a primary cell or an established cell line comprising embryonic stem cells or mesenchymal stem cells. In the method (3) or method (4), the embryonic stem cells or mesenchymal stem cells are preferably ones having an in vivo physiological function. Furthermore, in the method (3), the differentiated cells are preferably insulin-producing cells or nerve cells.

In another aspect, the invention provides

an antibody

which recognizes, as an antigen, the protein of the invention or the protein encoded by the novel gene.

In a further aspect, the invention provides

a method of diagnosing a disease

which utilizes said antibody. As the disease to be diagnosed by the diagnostic method of the invention, there may be mentioned diseases involving proliferative disease, pancreatic diseases, nervous system diseases, persistent hyperinsulinemic hypoglycemia of infancy, etc.

### DETAILED DESCRIPTION OF THE INVENTION

In the following, the present invention is described in detail.

In searching for a gene specifically expressed in proliferating insulin-producing cells or pancreatic β cells, the present inventors selected the pancreases of patients with persistent hyperinsulinemic hypoglycemia of infancy (PHHI) as tissues in which the expression of such a gene is highly possible. PHHI is a human hereditary disease also called nesidioblastosis and is known to involve a partial mutation of the potassium channel on pancreatic β cells. Therefore, the pancreatic β cells of patients with this disease are always stimulated to secrete insulin and the patients show severe symptoms of hypoglycemia (Science, 268, 426 (1995)). Furthermore, in PHHI patients, not only high blood insulin concentrations but also hyperplasia of islets of Langerhans, especially of pancreatic β cells, is observed, and such cells are clearly distinguishable from transformed cells such as cancer cells. Therefore, the pancreases of PHHI patients are regarded as a model of spontaneous proliferation of pancreatic β cells.

Therefore, the present inventors considered that if a gene specifically expressed in the pancreas of a PHHI patient could be identified, it would serve as a marker for detecting proliferating pancreatic β cells and, further, could code for a molecule causing the differentiation of precursor cells into pancreatic β cells or the proliferation of pancreatic β cells. Thus, they extracted RNA from the pancreases of PHHI patients and from the pancreases of normal subjects, synthesized cDNAs of the genes expressed in the respective tissues, performed gene subtraction using them, and successfully obtained novel genes specifically expressed in pancreases of PHHI patients.

Thus, the present invention relates to a novel gene specifically expressed in the pancreases of PHHI patients and the protein translated from the gene, and use thereof.

As the DNA of the invention, there may be mentioned

(1) a DNA

which codes for the protein comprising the amino acid sequence shown under SEQ ID NO:1, 2 or 3,

(2) a DNA

which comprises the base sequence shown under SEQ ID NO:4, 5 or 6, or

(3) a DNA which comprises the base sequence from the 174th to 904th base in SEQ ID NO:4, a DNA which comprises the base sequence from the 79th to 2115th base in SEQ ID NO:5, or a DNA which comprises the base sequence from the 28th to 384th base in SEQ ID NO:6, or

(4) a DNA comprising part of the base sequence shown under SEQ ID NO:4, 5 or 6

which has at least the base sequence of DNA of the partial sequence region defined above under (3).

Here, the DNAs comprising the base sequence shown under SEQ ID NO:4, 5, or 6 as defined above under (2) are all novel genes found in the process of completion of the present invention. These novel genes could be obtained in the following manner.

RNA is extracted from each of PHHI patients' pancreases and normal subjects' pancreases by the acidic phenol method and, after purification of polyA(+) RNA, cDNAs originating from the respective tissues are synthesized using reverse transcriptase. Using these cDNAs as materials, gene subtraction is carried out by the method of Hubank and Schatz (Nucleic Acids Res., 22, 5640 (1993)), and the genes specifically expressed in the pancreases of PHHI patients are detected. This time, the base sequences of the specific genes were determined and searched for through base sequence data bases, and it was confirmed that at least three of the specific genes are novel genes, namely genes whose function is unknown.

The three genes confirmed to be novel genes as a result of database searching were respectively designated as NC1, NC2 and NC3. The base sequences thereof are shown under SEQ ID NO:4 (NC1), SEQ ID NO:5 (NC2) and SEQ ID NO:6 (NC3) The amino acid sequences of the proteins translated from those genes as deduced from the base sequences are shown under SEQ ID NO:1 (NC1), SEQ ID NO:2 (NC2) and SEQ ID NO:3 (NC3).

In the practice of the present invention, a DNA covering a part of the base sequence of the novel gene can also be utilized for the purposes mentioned later herein. The DNA covering a part of the base sequence, so referred to herein, is not particularly restricted if the intended purposes can be achieved. Specifically, however, there may be mentioned a DNA comprising the base sequence from the 174th to 904th base in SEQ ID NO:4, a DNA comprising the base sequence from the 79th to 2115th base in SEQ ID NO:5, or a DNA comprising the base sequence from the 28th to 384th base in SEQ ID NO:6 and, further, a DNA comprising a part of the base sequence shown under SEQ ID NO:4, 5 or 6 and containing said partial sequence region.

The method of preparing the DNA of the invention is not particularly restricted but the gene obtained by the above-mentioned method of obtaining the novel gene may be used as such or only a part thereof may be used. A DNA having said sequence may also be prepared by chemical synthesis.

As the protein of the invention, there may be mentioned (1) a protein which comprises the amino acid sequence shown under SEQ ID NO:1, 2 or 3 and (2) a protein which has the amino acid sequence shown under SEQ ID NO:1, 2 or 3. The method for obtaining these proteins is not particularly restricted but the proteins can be obtained, for example, in the manner of genetic engineering by using Escherichia coli, animal cells or the like as the host, transforming the same with a vector containing the above-mentioned DNA of the invention as inserted therein, and cultivating the resulting transformant. As for the method of insertion into the vector, the method of transformation, the method of cultivation, etc., the respective methods known in the art can be used.

When a DNA derived from a gene specifically expressed in the pancreases of PHHI patients and the protein translated from that DNA are used in accordance with the invention, proliferating insulin-producing cells/pancreatic β cells can be detected and selected from a tissue or cell population comprising various cell species by such methods as mentioned below.

RNA is extracted from a target tissue or cell population and subjected to northern analysis using, as a probe, the specific gene-derived DNA of the invention as labeled by an appropriate method, for example with the radioisotope ³²P and a DNA-modifying enzyme. If proliferating insulin-producing cells/pancreatic β cells are present in the target tissue or cell population, they are detected upon autoradiography. It is also possible to detect proliferating insulin-producing cells/pancreatic β cells by carrying out PCR using cDNA synthesized based on the RNA extracted from the target tissue or cell population as a template and the DNA of the invention as a primer.

Furthermore, it is possible to detect proliferating insulin-producing cells/pancreatic β cells from the target tissue or cell population by an immunological technique, for example by tissue immunostaining, using an antibody prepared in advance and recognizing the protein of the invention as an antigen. This antibody can also be used in diagnosing a disease in which the proliferation of insulin-producing cells/pancreatic β cells is involved. As such diseases, there may be mentioned, for example, diseases involving proliferative disease, pancreatic diseases, nervous diseases and the like. In particular, the above antibody is judiciously used in diagnosing persistent hyperinsulinemic hypoglycemia of infancy, among others.

Since the gene of the invention is specifically expressed in PHHI patients' pancreases, which is a model of spontaneous proliferation of pancreatic β cells, it is presumable that it is involved in the proliferation or differentiation of pancreatic β cells or cells related thereto. Therefore, when the DNA of the invention derived from said gene is introduced into insulin-producing cells/pancreatic β cells, into cells related thereto and precursor cells thereof, for example nerve cells or the like, or into cultured cells corresponding to such precursor cells, for example embryonic stem cells, mesenchymal stem cells or the like, and caused to be expressed, the proliferation of such cells can expectedly be promoted. Similarly, when the DNA of the invention is introduced into insulin-producing cells/pancreatic β cells, into cells related thereto and precursory thereof, for example nerve cells or the like, or into cultured cells corresponding to such precursor cells, for example embryonic stem cells, mesenchymal stem cells or the like, and caused to be expressed, the differentiation of such cells into insulin-producing cells or nerve cells can expectedly be induced. These embryonic stem cells or mesenchymal stem cells are preferably ones having an in vivo physiological function. The in vivo physiological function, so referred to herein, means a high level of in vivo or in vitro differentiating ability. As regards the method of introduction and expression, any of those known methods which are in general use can be employed.

### BRIEF DESCRIPTION OF THE DRAWINGS

FIG. 1 is a representation of the results of investigation, by northern analysis, of the expression of the NC1, NC2 and NC3 genes specific to the pancreases of patients with persistent hyperinsulinemic hypoglycemia of infancy.

FIG. 2 is a representation of the results of investigation, by northern analysis, of the changes in expression of the NC3 gene as resulting from cell differentiation.

FIG. 3 is a representation of the changes in morphology of PC12 cells after forced expression of the NC1 gene.

### BEST MODE FOR CARRYING OUT THE INVENTION

In the following, the present invention is described more specifically by means of examples. However, these examples are no limitative of the present invention without departing from the scope of the invention.

### EXAMPLE 1

### Method for Obtaining the Novel Genes NC1, NC2 and NC3

All RNA was extracted from a PHHI patient's pancrease and a normal subject's pancrease using RNAzolB (product of Biotecx Laboratories Inc.) to purify polyA(+) RNA using PolyATract messenger RNA isolation system (product of Promega). From this polyA(+) RNA, double strand cDNAs were synthesized using Riboclone cDNA Synthesis System (product of Promega). Each of the double strand cDNAs derived from the PHHI patient's pancrease and the normal subject's pancrease was completely cut by restriction enzyme DpnII (product of New England Biolabs Inc.). After stopping the reaction by phenol treatment, the cut cDNA fragments were recovered by ethanol precipitation. The recovered DNA fragments were suspended in sterilized distilled water, and 8 μg of R-Bgl-24 (oligo DNA having the base sequence of agcactctccagccctctcaccgca) and 4 μg of R-Bgl-12 (gatctgcggtga) were ligated to 1.2 μg of the cDNA fragment at 14° C. for 16 hours using T4 DNA ligase (product of New England Biolabs Inc.) with a scale of 50 μl. The DNA concentration in the reaction product was adjusted to 6 μg/ml, and 1 μl of the product was fractionated and amplified by PCR reaction using R-Bgl-24 as a primer. The amplification product derived from the PHHI patient's pancrease is called as a tester, and the amplification product derived from the normal subject is called as a driver. The tester and driver were cut by DpnII to remove R-Bgl-24 and R-Bgl-12 oligo DNA, and to the tester alone, other oligo DNA J-Bgl-24 (accgacgtcgactatccatgaaca) and J-Bgl-12 (gatctgttcatg) were ligated. To 0.4 μg of the tester to which the oligo DNA has been ligated, centuple amount (40 μg) of the driver was added, and ethanol precipitation was carried out. Thereafter, the obtained product was suspended in 4 μl of EE×3 buffer solution [30 mM N-(2-hydroxy-ethyl)piperazine-N′-3-propane sulfonic acid (product of Sigma); 3 mM EDTA (pH 8.0)]. This DNA solution was denatured by heat treatment at 98° C. for 5 minutes, kept at 67° C. for 20 hours for annealing, and a TE buffer solution [10 mM Trizma Base (product of Sigma); 1 mM EDTA (pH 8.0)] was added so as the total amount to be 400 μl. 10 μl of the mixture solution was fractionated, and kept at 72° C. for 3 minutes to detach J-Bgl-12 from the cDNA. 5 units of Taq polymerase (product of New England Biolabs Inc.) were added thereto, and the resultant was further subjected to reaction for 5 minutes to modify the single strand part to a double strand. Using this reaction product as a mold, 10 cycles of PCR reaction was carried out at 95° C. for 1 minute/70° C. for 3 minutes. The amplification product was subjected to phenol treatment and ethanol precipitation, and suspended in 400 μl of 0.2-fold TE buffer solution. 40 units of Mung Bean Nuclease (product of New England Biolabs Inc.) were added thereto, and the resultant was subjected to reaction at 30° C. for 35 minutes. This reaction product is called as DP1. 2 μg of J-Bgl-24 was added to 10 μl of DP1 as a primer, and the resultant was subjected to 18 cycles of PCR reaction at 95° C. for 1 minute/70° C. for 3 minutes. The obtained amplification product was cut by DpnII, 8 μg of N-Bgl-24 (aggcaactgtgctatcgagggaa) and 4 μg of N-Bgl-12 (gatcttccctcg) were ligated to 1.2 μg of the cDNA fragment, and the process for obtaining DP1 was followed to obtain DP2. DP2 was amplified by PCR reaction using N-Bgl-24 as a primer. The amplification product was cut by DpnII and ligated to J-Bgl-24 and J-Bgl-12 to obtain DP3 in the same manner. DP3 was amplified by PCR reaction (95° C. for 1 minute/70° C. for 3 minutes, 22 cycles) using J-Bgl-24 as a primer. The obtained amplification product was cut by DpnII, and then the resultant was subcloned to BamHI site of pUC19. The base sequence of the obtained clone was determined, and the base sequence data bases such as Gen Bank were searched. As a result, three of those clones were not identical to various base sequences on the data bases. Furthermore, using these clones as probes, the cDNA library was screened to obtain full-length cDNA, and designated as NC1, NC2 and NC3.

### EXAMPLE 2

### Detection of Proliferating Insulin Cells/Pancreatic β Cells by Northern Analysis

Using a part of the DNA in the gene sequences of NC1 (SEQ ID NO:4), NC2 (SEQ ID NO:5), and NC3 (SEQ ID NO:6) obtained in Example 1, whether the objective tissues contain proliferating insulin-producing cells or not was investigated by Northern analysis.

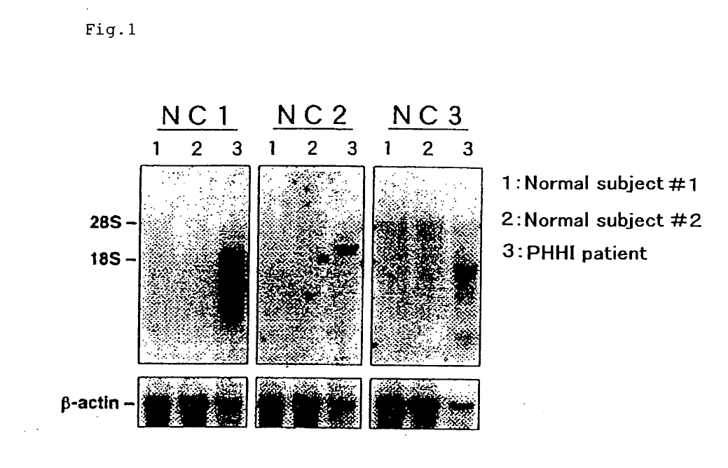

All RNA was extracted from normal subjects' pancreases (lane 1 and lane 2, two samples) and a PHHI patient's pancreas (lane 3, one sample) using TRIzol (product of GIBCO Lifetech Oriental), and then purified. After fractionating these by 1.0% agarose gel electrophoresis, the resultant was transferred onto a nylon membrane (Hybond-N, product of Amersham Pharmacia Biotech Inc.) by a capillary method. A DNA comprising the base sequence from the 174th to 904th base in SEQ ID NO:4, a DNA comprising the base sequence from the 79th to 2115th base in SEQ ID NO:5, and a DNA comprising the base sequence from the 28th to 384th base in SEQ ID NO:6 were amplified by a PCR method. After fractionating the resultant by agarose gel electrophoresis, the objective DNA fragment was cut from the gel and purified. These were radiolabeled using T4 polynucleotide kinase (product of TAKARA SHUZO CO., LTD) and [γ-32P]ATP (product of Amersham Pharmacia Biotech Inc.), and used as a probe for Northern analysis. The RNA-transferred nylon membrane and the radiolabeled probe were mixed and hybridized overnight at 65° C., the membrane was washed, and an RNA fraction reactive with the probe was detected by autoradiography (FIG. 1). When the DNAs of the partial sequences derived from NC1, NC2 and NC3 were used as probes, although a signal was detected from RNA derived from the PHHI patient, no signal was detected from RNA derived from the normal subject.

### EXAMPLE 3

### Changes in Expression of NC3 as Resulting from Cell Differentiation

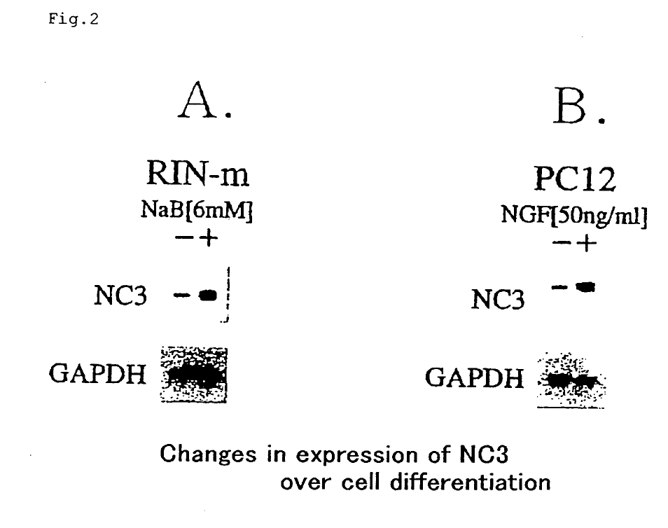

Although the insulin-producing ability of RIN-m cells established from rat insulinoma are low, it has been shown that the cells become to secrete insulin at high concentration by adding sodium butyrate to a cultivation system, and are differentiated into insulin-producing cells (Bartholomeusz et al., Endocrinology vol. 24, 2680-2685 pages, 1989). After RIN-m cells were cultivated in the presence of 6 mM sodium butyrate for 16 hours, all RNA was extracted, and Northern analysis was carried out in the same manner as described in Example 2 using a DNA comprising the base sequences from the 28th to 384th base in SEQ ID:6 as a probe. As a result, as shown in A of FIG. 2, the expression of NC3 gene was promoted in RIN-m cells which have been cultivated by adding sodium butyrate.

Furthermore, PC-12 cells derived from adrenal pheochromocytoma are known to elongate the neurite by adding nerve growth factor (NGF), and to differentiate into nerve cell like (Saltiei et al., Bioessay vol.16, 405-411 pages, 1994). All RNA was extracted from PC-12 cells which have been cultivated for 16 hours in the cultivation system added with NGF to have a concentration of 50 ng/ml. A DNA comprising the base sequence from the 28th to 384th base in SEQ ID NO:6 was used as a probe, and Northern analysis was carried out in the same manner. As shown in B of FIG. 2, the expression of NC3 gene was promoted in PC-12 cells which have been cultivated with an addition of NGF.

### EXAMPLE 4

### Changes in Morphology of PC12 Cells after Forced Expression of NC1 Gene

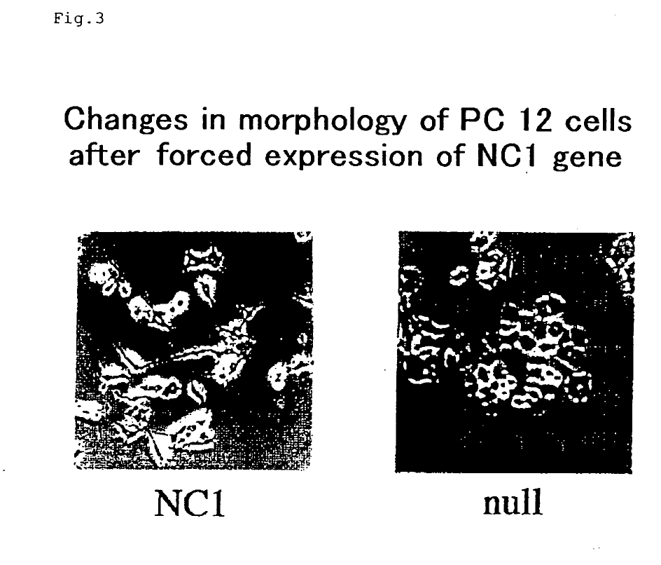

Among the base sequences shown under SEQ ID NO:4, a DNA of the base sequence from the 174th to 904th base, which is a region coding for a protein comprising the amino acid sequence of the SEQ ID NO:1, was inserted into a predetermined site of pCIneo, which is a gene expression vector for animal cells, and thereby an NC1 gene expression vector was constructed. This was introduced into PC-12 cells by a liposome method to carry out forced expression. The cells were kept in the cultivation system to which geneticin has been added at a concentration of 500 μg/ml, and the cells having the expression vector were selected. As shown in FIG. 3, cells elongating neurites were observed in NC1 gene expression-forced PC-12 cells, and it was suggested that PC-12 cells could be differentiated into nerve cell like by forced expression of NC1 gene.

### INDUSTRIAL APPLICABILITY

The novel genes found in the present invention are not only specifically expressed in PHHI patients' pancreases, but also changing its expression over cell differentiation and/or proliferation, and capable of inducing differentiation into cells having a function by forcibly expressing into undifferentiated cells in genetic engineering manner. Accordingly, it becomes possible to easily detect insulin-producing cells capable of proliferating by using these genes, a part of the DNA and a protein translated therefrom, to select them, and further to differentiate and proliferate insulin-producing cells. Furthermore, it becomes possible to develop a novel diagnostic method of various diseases resulting from abnormal differentiation and proliferation of pancreatic β cells, which are insulin-producing cells, and cells related to pancreatic β cells (for example nerve cells).

1. A protein
which consists of the amino acid sequence shown under SEQ ID NO:1.

2. A protein
which comprises the amino acid sequence shown under SEQ ID NO:1.

3. A DNA
which codes for the protein comprising the amino acid sequence shown under SEQ ID NO:1:

4. A DNA
which comprises the base sequence shown under SEQ ID NO:4.

5. A DNA
which comprises the base sequence from the 174th to 904th base in SEQ ID NO:4.

6. A DNA
which has at least the base sequence from the 174th to 904th base in SEQ ID NO:4.

7. A method of detection of a proliferating insulin-producing cell
which comprises using at least one species of the DNA selected from the group consisting of a DNA coding for the protein comprising the amino acid sequence shown under SEQ ID NO:1, 2 or 3, a DNA comprising the base sequence shown under SEQ ID NO:4, 5 or 6, a DNA comprising the base sequence from the 174th to 904th base in SEQ ID NO:4, a DNA comprising the base sequence from the 79th to 2115th base in SEQ ID NO:5, a DNA comprising the base sequence from the 28th to 384th base in SEQ ID NO:6, a DNA having at least the base sequence from the 174th to 904th base in SEQ ID NO:4, a DNA having at least the base sequence from the 79th to 2115th base in SEQ ID NO:5 and a DNA having at least the base sequence from the 28th to 384th base in SEQ ID NO:6.

8. A transformant cell
which is obtainable by using a vector containing the DNA according to any one of claims 3 to 6 inserted therein.

9. A method of differentiation of an embryonic stem cell or a mesenchymal stem cell
which comprises introducing at least one species of the DNA selected from the group consisting of a DNA coding for the protein comprising the amino acid sequence shown under SEQ ID NO:1, 2 or 3, a DNA comprising the base sequence shown under SEQ ID NO:4, 5 or 6, a DNA comprising the base sequence from the 174th to 904th base in SEQ ID NO:4, a DNA comprising the base sequence from the 79th to 2115th base in SEQ ID NO:5, a DNA comprising the base sequence from the 28th to 384th base in SEQ ID NO:6, a DNA having at least the base sequence from the 174th to 904th base in SEQ ID NO:4, a DNA having at least the base sequence from the 79th to 2115th base in SEQ ID NO:5 and a DNA having at least the base sequence from the 28th to 384th base in SEQ ID NO:6, into primary cells or established cell lines having an embryonic stem cell or a mesenchymal stem cell.

10. A method of proliferation of an embryonic stem cell or a mesenchymal stem cell
which comprises introducing at least one species of the DNA selected from the group consisting of a DNA coding for the protein comprising the amino acid sequence shown under SEQ ID NO:1, 2 or 3, a DNA comprising the base sequence shown under SEQ ID NO:4, 5 or 6, a DNA comprising the base sequence from the 174th to 904th base in SEQ ID NO:4, a DNA comprising the base sequence from the 79th to 2115th base in SEQ ID NO:5, a DNA comprising the base sequence from the 28th to 384th base in SEQ ID NO:6, a DNA having at least the base sequence from the 174th to 904th base in SEQ ID NO:4, a DNA having at least the base sequence from the 79th to 2115th base in SEQ ID NO:5 and a DNA having at least the base sequence from the 28th to 384th base in SEQ ID NO:6, into a primary cell or an established cell line having an embryonic stem cell or a mesenchymal stem cell.

11. The method according to claim 9 or 10,
wherein the embryonic stem cell or mesenchymal stem cell has an in vivo physiological function.

12. The method according to claim 9,
wherein the differentiated cell is an insulin-producing cell.

13. The method according to claim 9,
wherein the differentiated cell is a nerve cell.

14. An antibody
which recognizes, as an antigen, any one of the following proteins (a) to (c):
(a) the protein according to claim 1;
(b) the protein according to claim 2; and
(c) the protein encoded by the DNA according to any one of claims 3 to 6.

15. A method of diagnosing a disease which utilizes the antibody according to claim 14.

16. The method of diagnosing a disease according to claim 15,
wherein the objective disease is a proliferative disease.

17. The method of diagnosing a disease according to claim 15,
wherein the objective disease is a pancreatic disease.

18. The method of diagnosing a disease according to claim 15,
wherein the objective disease is a nervous system disease.

19. The method of diagnosing a disease according to claim 15,
wherein the objective disease is persistent hyperinsulinemic hypoglycemia of infancy.
